(19) United States
(12) Patent Application Publication
(10) Pub. No.: US 2006/0068424 A1
(43) Pub. Date: Mar. 30, 2006

(54) Biosensor

(75) Inventors: Toshiaki Kubo, Kanagawa (JP); Koichi Kawamura, Shizuoka (JP); Takeyoshi Kano, Shizuoka (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/202,138

(22) Filed: Aug. 12, 2005

(30) Foreign Application Priority Data
Aug. 13, 2004 (JP) 236071/2004
Aug. 13, 2004 (JP) 236072/2004

(51) Int. Cl. C12Q 1/68; C12M 1/34

(52) U.S. Cl. 435/6; 435/287.2

(57) ABSTRACT

An object of the present invention is to provide a biosensor wherein at least two types of surfaces are patterned without impairing the sensor surface and non-specific adsorption is thereby suppressed. The present invention provides a biosensor comprising a substrate coated with a hydrophobic compound having a photoactive group, or a substrate which is coated with a hydrophobic polymer and is further modified with a compound having a photoactive group.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The OCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (run level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

When a specific binding reaction is measured between a physiologically active substance and a test substance, the test substance does not necessarily consist of a single component, but it is sometimes required to measure the test substance existing in a heterogeneous system, such as in a cell extract. In such a case, if various contaminants such as proteins or lipids were non-specifically adsorbed on the detection surface, detection sensitivity in measurement would significantly be decreased. The aforementioned detection surface has been problematic in that such non-specific adsorption often takes place thereon. In order to solve such a problem, several methods have been studied. For example, a method of immobilizing hydrophilic hydrogel on a metal surface via a linker, so as to suppress physical adsorption, has been applied (Japanese Patent No. 2815120, U.S. Pat. No.5,436,161, and Japanese Patent Laid-Open No. 8-193948). However, the ability to suppress non-specific adsorption of this method has not yet been sufficient.

In order to eliminate influence of measurement disturbance (changes in temperature, concentration, and pressure) thereby reducing baseline fluctuation, a measurement unit for measuring a specific binding reaction between a physiologically active substance and a test substance and a reference unit wherein such a binding reaction is not carried out preferably exist on a single plane of the above-described biosensor, and are located as close as possible to each other. Thus, it became necessary to allow a reference unit and a measurement unit to coexist on an SPR sensor surface using a thin polymer film.

U.S. Pat. No. 6,444,254 describes a method for microstamping a polymer surface with a biological ligand, which comprises: forming a first functional group on a polymer surface by at least one method selected from the group consisting of hydrolysis, reduction, photoinitiated graft polymerization, amination, a surface cross-polymerization of polyethylene oxide, a chemical reaction of a terminal hydroxyl group, corona discharge, plasma etching, laser treatment, and ion beam treatment; allowing a stamp, on which at least one biological ligand having a second functional group has been adsorbed, to come into contact with the above surface, so as to form a covalent bond with the first functional group on the polymer surface; and separating the stamp from the polymer surface, so as to directly immobilize the biological ligand on the polymer surface via a covalent bond. In the aforementioned method, a solid (PDMS) is allowed to come into contact with a polymer film for patterning. However, since a sensor used for SPR has a surface formed by adding a thin polymer film onto a thin metal film, the physical strength of the thus formed surface is low. Thus, a sensor surface would be damaged, if a solid were allowed to come into contact therewith. Accordingly, the aforementioned method is not suitable for SPR.

### DISCLOSURE OF INVENTION

It is an object of the present invention to solve the aforementioned problems. In particular, it is an object of the present invention to provide a biosensor wherein at least two types of surfaces are patterned without impairing the sensor surface and non-specific adsorption is thereby suppressed.

As a result of intensive studies directed towards achieving the aforementioned object, the present inventors have found that a desired biosensor can be provided by coating the substrate surface with a hydrophobic compound having a photoactive group and performing patterning by light irradiation. They have found also that a desired biosensor can be provided by allowing a compound having a photoactive group to come into contact with a substrate coated with a hydrophobic polymer and then performing patterning by light irradiation. The present invention has been completed based on these findings.

Thus, the present invention provides a biosensor comprising a substrate coated with a hydrophobic compound having a photoactive group, or a substrate which is coated with a hydrophobic polymer and is further modified with a compound having a photoactive group.

Preferably, at least two types of surfaces are patterned by light irradiation on a substrate.

Preferably, the substrate is a metal surface or metal film.

Preferably, the metal surface or metal film consists of a free electron metal selected from the group consisting of gold, silver, copper, platinum, and aluminum.

Preferably, the thickness of the metal film is between 0.1 nm and 500 nm.

Preferably, the coating thickness of the hydrophobic compound having a photoactive group or the hydrophobic polymer is between 0.1 nm and 500 nm.

Preferably, the biosensor of the present invention has a functional group capable of immobilizing a physiologically active substance on the outermost surface of the substrate.

Preferably, the compound having a photoactive group has a functional group capable of immobilizing a physiologically active substance.

Preferably, the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R²and R³ independently represents a hydrogen atom or lower alkyl group), —NCO, —NCS, an epoxy group, or a vinyl group.

Preferably, the biosensor of the present invention has a functional group capable of immobilizing a physiologically active substance in a certain region on the outermost surface of the substrate, which has been patterned by light irradiation.

Preferably, the biosensor of the present invention is used in non-electrochemical detection, and more preferably in surface plasmon resonance analysis.

Preferably, the biosensor of the present invention is formed in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface,

wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

In another aspect, the present invention provides a method for producing the aforementioned biosensor of the present invention, which comprises a step of coating a substrate with a hydrophobic compound having a photoactive group and a step of applying light to the substrate.

In further another aspect, the present invention provides a method for producing the aforementioned biosensor of the present invention, which comprises a step of coating a substrate with a hydrophobic compound having a photoactive group, a step of allowing a monomer compound having a functional group capable of immobilizing a physiologically active substance to come into contact with the substrate, and a step of applying light to only certain region of the substrate with making patterning so as to bind said monomer compound.

In further another aspect, the present invention provides a method for producing the aforementioned biosensor of the present invention, which comprises a step of allowing a substrate coated with a hydrophobic polymer to come into contact with a compound having a photoactive group.

In further another aspect, the present invention provides a method for producing the aforementioned biosensor of the present invention, which comprises a step of allowing a compound having a photoactive group to come into contact with a substrate coated with a hydrophobic polymer, and a step of applying light to only certain region of the substrate with making patterning.

In further another aspect, the present invention provides the biosensor according to the present invention, wherein a physiologically active substance is bound to the surface by covalent bonding.

Preferably, the present invention provides the biosensor mentioned above wherein a physiologically active substance is bound by using a compound having a photoactive group.

Preferably, at least one measurement unit to which a physiologically active substance or a substance interacting therewith binds, and one reference unit that does not have a physiologically active substance or a substance interacting therewith, exist on a single plane.

Another aspect of the present invention provides a method for immobilizing a physiologically active substance on a biosensor, which comprises a step of allowing a physiologically active substance to come into contact with the biosensor according to the present invention, so as to allow said physiologically active substance to bind to the surface of said biosensor via a covalent bond.

Another aspect of the present invention provides a method for detecting or measuring a substance interacting with a physiologically active substance, which comprises a step of allowing a test substance to come into contact with the biosensor according to the present invention to the surface of which the physiologically active substance binds via a covalent bond.

Preferably, the substance interacting with the physiologically active substance is detected or measured by a non-electrochemical method. More preferably, the substance interacting with the physiologically active substance is detected or measured by surface plasmon resonance analysis.

### BRIEF DESCRIPTION OF THE DRAWINGS

FIG. 1 shows the surface plasmon resonance measurement device used in the present invention.

### BEST MODE FOR CARRYING OUT THE INVENTION

The embodiments of the present invention will be described below.

The first embodiment of the biosensor of the present invention is characterized in that it comprises a substrate coated with a hydrophobic compound having a photoactive group. More preferably, the biosensor of the present invention is characterized in that at least two types of surfaces are patterned by applying light to a substrate coated with a hydrophobic compound having a photoactive group.

The second embodiment of the biosensor of the present invention is characterized in that it comprises a substrate which is coated with a hydrophobic polymer and is further modified with a compound having a photoactive group. More preferably, the biosensor of the present invention is characterized in that at least two types of surfaces are patterned by applying light to a substrate.

(1) Hydrophobic Compound Having a Photoactive Group

A hydrophobic compound having a photoactive group used in the first embodiment of the present invention will be described. Examples of a method for obtaining a hydrophobic compound having a photoactive group used in the present invention may include: (1) a method of introducing a photoactive group or a compound having a photoactive group into a common hydrophobic compound; (2) a method of introducing a hydrophobic group into a polymer having a photoactive group; and (3) a method of mixing a compound having a photoactive group into a hydrophobic compound.

Examples of a common hydrophobic compound used in the method of introducing a photoactive group or a compound having a photoactive group into a common hydrophobic compound described in (1) above may include compounds having an aromatic group or alkyl group.

In addition, application of products obtained by polymerization of the below-mentioned hydrophobic monomers is also a preferred embodiment. Examples of such a hydrophobic monomer may include monomers having a hydrophobic group such as (meth)acrylate or styrene. A homopolymer obtained using at least one selected from these hydrophobic monomers, a copolymer obtained by copolymerization of two or more selected therefrom, or the like, may be selected depending on purpose. In the case of copolymerization, either a random copolymerization or a block copolymerization may be applied.

The photoactive group used in the present invention indicates a group which is decomposed by light irradiation with an area ranging from 150 nm to 1,200 nm, so as to generate radicals. Specific examples of such a photoactive group may include an azide group generating nitrene by light irradiation, a diazo group generating radicals by light irradiation, a benzophenone group, a trichloromethyl group, an α-hydroxyacetophenone group, and an α-alkoxyacetophenone group. Examples of a compound having such a photoactive group may include an azide compound, a diazo compound, and a trichloromethyl-triazine compound.

Examples of a polymer having a photoactive group that is used in the method of introducing a hydrophobic group into a polymer having a photoactive group described in (2) above may include a diazo compound, an azide compound, and a benzophenone compound. Examples of a hydrophobic group introduced into a polymer having the aforementioned photoactive group may include (meth)acrylate and styrene.

Examples of a compound having a photoactive group and a hydrophobic compound that are used in the method of mixing a compound having a photoactive group into a hydrophobic compound described in (3) above are the same as those described for the aforementioned methods (1) and (2).

The composition ratio between a hydrophobic group and a photoactive group contained in the hydrophobic compound having the photoactive group synthesized by the methods described in (1) and (2) above, is preferably between 1:99 and 90:10, and more preferably between 5:95 and 60:40. Such a composition ratio can be controlled by adjusting the amount of a photoactive group added to the aforementioned common hydrophobic compound or the amount of a hydrophobic group added to a polymer having a photoactive group during the synthesis.

When a photoactive group or a compound having such a photoactive group used in the present invention is introduced into a hydrophobic compound having a polymer structure obtained by the aforementioned polymerization of hydrophobic monomers, the position in which the above photoactive group or compound is introduced may be a side chain and/or a terminus. From the viewpoint of expression of hydrophobicity, such position may preferably be a terminus. Specific examples of hydrophobic compounds having photoactive groups (hydrophobic compound 1 to hydrophobic compound 15) are given below, together with composition ratios thereof. However, the present invention is not limited to such examples.

Hydrophobic Compound 1˜15

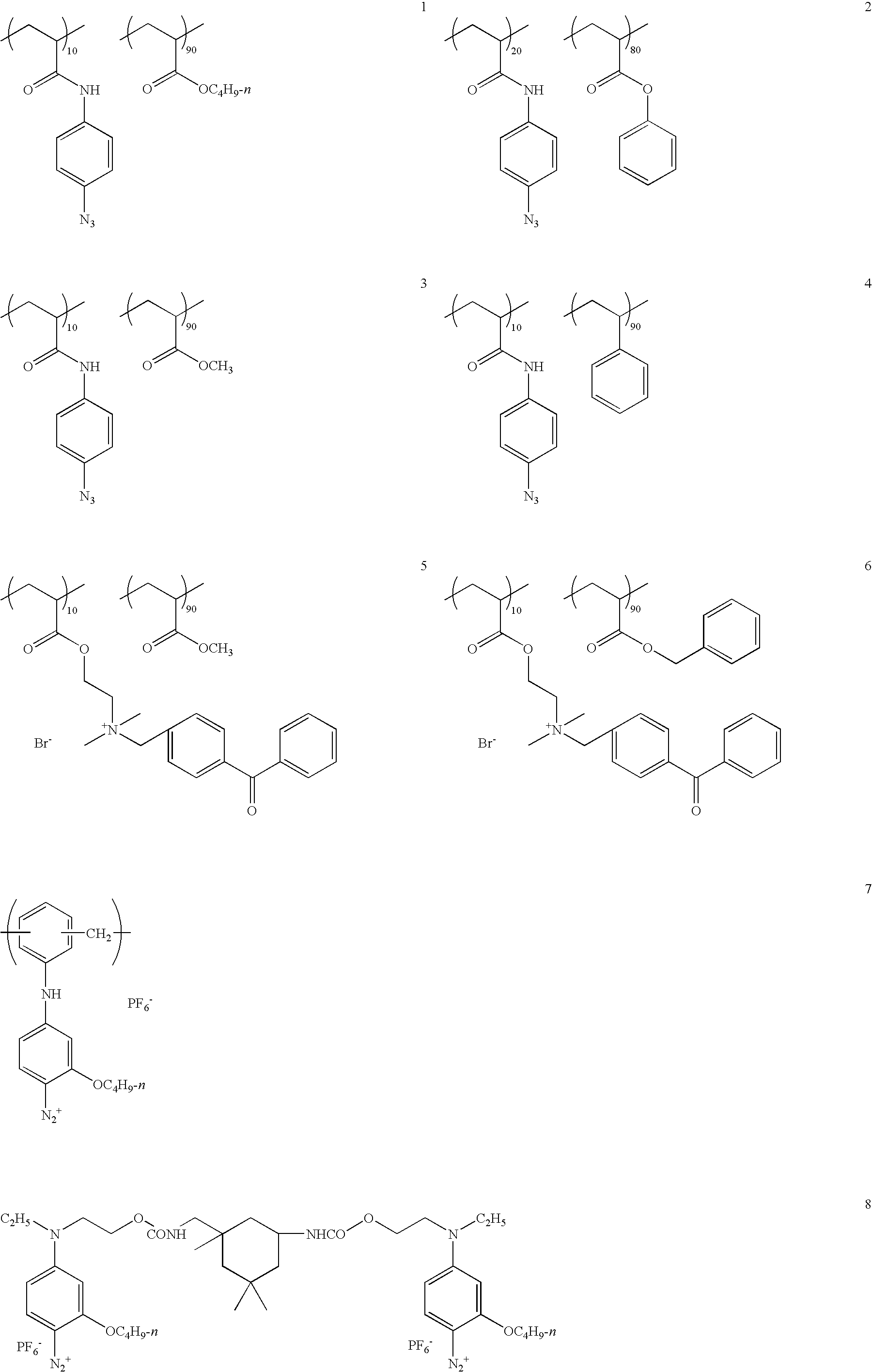

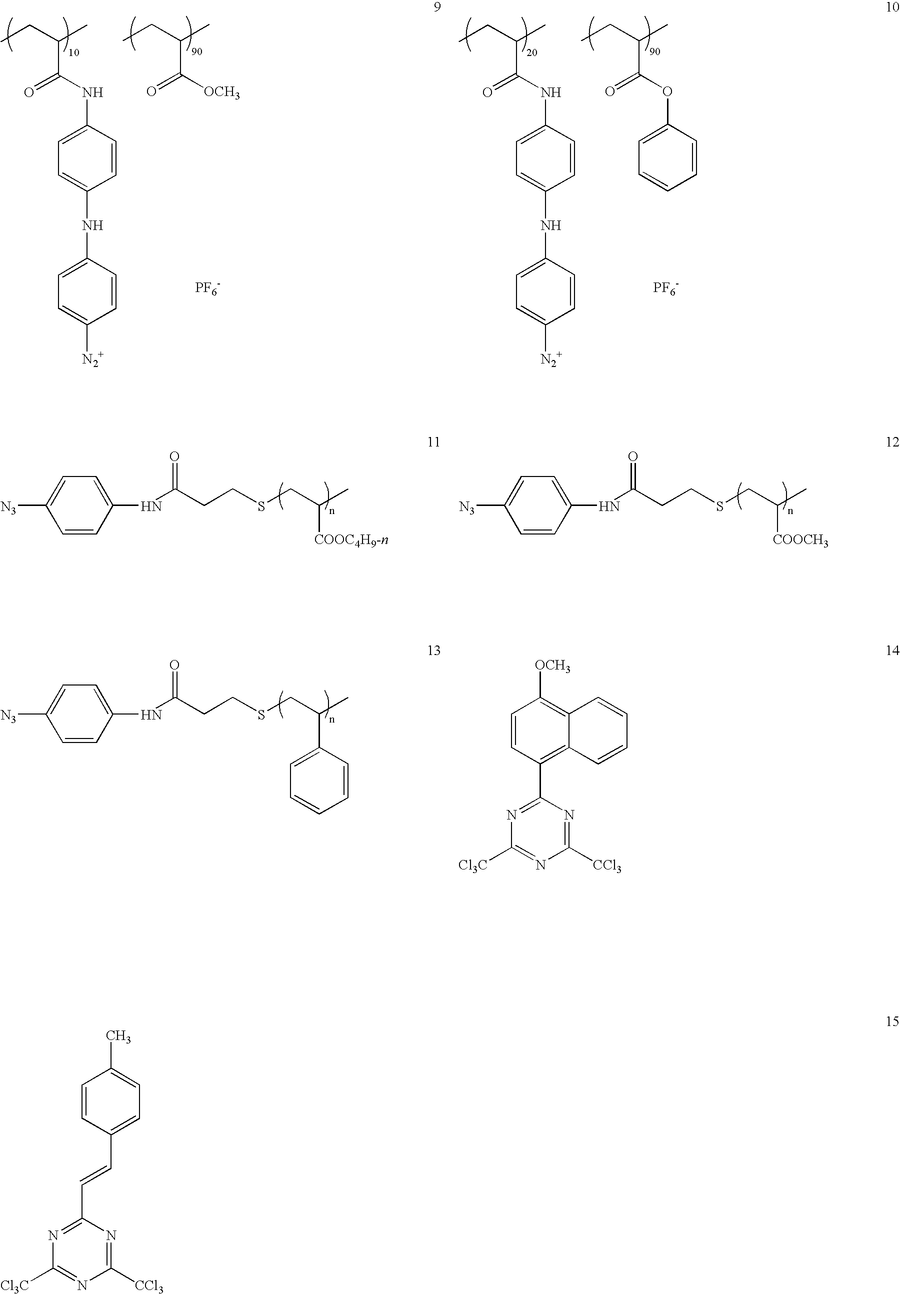

Such a hydrophobic compound having a photoactive group can be synthesized by a known method, as described in Japanese Patent Application Laid-Open (Kokai) No. 2003-345038.

A substrate can be coated with a hydrophobic compound having a photoactive group according to common methods. Examples of such a coating method may include spin coating, air knife coating, bar coating, blade coating, slide coating, curtain coating, spray method, evaporation method, cast method, and dip method.

The coating thickness of a hydrophobic compound having a photoactive group is not particularly limited, but it is preferably between 0.1 nm and 500 nm, and particularly preferably between 1 nm and 300 nm.

(2) Hydrophobic Polymer and Compound Having a Photoactive Group

The hydrophobic polymer used in the second embodiment of the present invention is a polymer having no water-absorbing properties. Its solubility in water (at 25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A substrate is coated with a hydrophobic polymer according to common methods. Examples of such a coating method may include spin coating, air knife coating, bar coating, blade coating, slide coating, curtain coating, spray method, evaporation method, cast method, and dip method.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ was calculated by the following formula. As the cohesive energy (Ecoh) of each functional group and the mol volume (V), those defined by Fedors were used (R. F. Fedors, Polym.Eng.Sci., 14(2), P147, P472(1974)).
Δ=(ΣEcoh/ΣV)^{1/2}

Examples of the SP values of the hydrophobic polymers and the solvents are shown below;
Solvent: 2-phenoxyethanol : 25.3 against polymethylmethacrylate-polystyrene copolymer (1:1):21.0
Solvent: acetonitrile: 22.9 against polymethylmethacrylate: 20.3
Solvent: toluene: 18.7 against polystyrene: 21.6

The period for contacting a substrate with a liquid which does not contain the hydrophobic polymer is not particularly limited, and is preferably 1 second to 24hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 0.1 nm and 500 nm, and particularly preferably between 1 nm and 300 nm.

Next, a compound having a photoactive group used in the second embodiment of the present invention will be described. The photoactive group used in the present invention indicates a group which is decomposed by light irradiation with an area ranging from 150 nm to 1,200 nm, so as to generate radicals. Specific examples of such a photoactive group may include an azide group generating nitrene by light irradiation, a diazo group generating radicals by light irradiation, a benzophenone group, a trichloromethyl group, an α-hydroxyacetophenone group, and an α-alkoxyacetophenone group. Examples of a compound having such a photoactive group may include an azide compound, a diazo compound, and a trichloromethyl-triazine compound. Specific examples of a compound having a photoactive group may include the following compounds, but examples are not limited thereto. 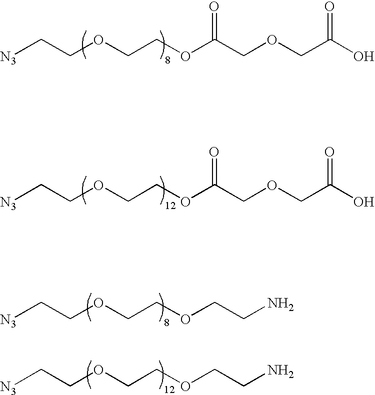
(3) Patterning

When a pattern is formed in the present invention, a method of energy transfer is not particularly limited. If radicals can be generated by decomposition of photoactive groups, either light exposure or heating can be applied. In terms of cost reduction and facilitation of a device, a method of applying active light is preferable. When irradiation with active light is applied for light exposure to images or the like, either scanning light exposure based on digital data, or pattern light exposure using a lith film can be applied. An example of a method for forming a pattern may be a method of writing by application of radiation such as heating or light exposure. Examples of such a method used herein may include: light irradiation using an infrared laser, an ultraviolet lamp, or a visible light; electron beam irradiation using γ ray or the like, and thermal recording using a thermal head. Examples of a light source used in these methods may include a mercury lamp, a metal halide lamp, a xenon lamp, a chemical lamp, and a carbon arc lamp. Examples of a radioactive ray may include an electron beam, X-ray, an ion beam, and far-infrared ray. Moreover, g ray, i ray, Deep-UV light, and high-density energy beam (laser beam) can also be used. Preferred examples of a commonly used embodiment may include direct image recording using a thermal recording head or the like, scanning light exposure using an infrared laser, high illuminance flash exposure using an xenon discharge lamp or the like, and infrared lamp exposure. In order to directly form a pattern using digital data of computers, it is preferable to transfer energy by laser exposure. Examples of a laser used herein may include: gas lasers such as a carbon gas laser, a nitrogen laser, an Ar laser, a He/Ne laser, a He/Cd laser, or a Kr laser; liquid (dye) lasers; solid lasers such as a ruby laser or a Nd/YAG laser; semiconductor lasers such as a GaAs/GaAlAs laser or an InGaAs laser; and excimer lasers such as a KrF laser, a XeCl laser, a XeF laser, or Ar₂. Of these, a semiconductor laser for applying an infrared ray with a wavelength from 700 to 1,200 nm, and a solid-state high-power infrared laser such as a YAG laser, are preferably used for light exposure. After such light exposure, the resultant product is washed with water, so as to dissolve and eliminate unexposed polymer portions.

In the present invention, at least two types of surfaces are patterned by light irradiation on a biosensor surface coated with a hydrophobic compound having a photoactive group, or a substrate which is coated with a hydrophobic polymer and is further modified with a compound having a photoactive group. Such patterning can be carried out, for example, to pattern a portion on which a physiologically active substance is immobilized and a portion on which such a physiologically active substance is not immobilized.

An example of at least two types of surfaces formed on a single plane may be a combination of a measurement unit to which a physiologically active substance or a substance interacting therewith binds, with a reference unit that does not have a physiologically active substance or a substance interacting therewith.

In the present invention, by establishing a measurement unit and a reference unit on a single plane as described above, baseline fluctuation caused by disturbance can be canceled, and it can substantially be stabilized.

In the first embodiment of the biosensor of the present invention, for example, a substrate is coated with a hydrophobic compound having a photoactive group, and a monomer compound having a functional group that is capable of immobilizing a physiologically active substance is then come into contact with the above substrate. Thereafter, while patterning is performed only in a certain region of the above substrate, light is applied thereto, so as to allow the above monomer compound to bind to the above substrate. A linker having a functional group capable of immobilizing a physiologically active substance is generated only in a region to which light has been applied, as a result of the above operation, but no linkers are generated in regions to which light has not been applied. After completion of the light irradiation, the substrate is washed with an appropriate solution (for example, water) to eliminate the monomer compound, so that the surface of the substrate can be patterned to a region having the functional group capable of immobilizing a physiologically active substance thereon and to a region that does not have such a functional group.

In the second embodiment of the biosensor of the present invention, for example, a substrate is coated with a hydrophobic compound, and thereafter, a compound having both a functional group that is capable of immobilizing a physiologically active substance and a photoactive group is come into contact with the above substrate. Thereafter, while patterning is performed only in a certain region of the above substrate, light is applied thereto, so as to allow the above compound having both the functional group and the photoactive group to bind to the above hydrophobic compound. By this operation, a region having a functional group capable of immobilizing a physiologically active substance is formed only in a region to which light has been applied. After completion of the light irradiation, the substrate is washed with an appropriate solution (for example, water) to eliminate an unbound compound (having both the functional group and the photoactive group), so that the surface of the substrate can be patterned to a region having the functional group capable of immobilizing a physiologically active substance and to a region that does not have such a functional group.

The type of a functional group capable of immobilizing a physiologically active substance is not particularly limited in the present specification. Examples of a preferred functional group may include —OH, —SH, —COOH, —NR¹R² (wherein each of R¹ and R² independently represents a hydrogen atom or a lower alkyl group), —CHO, —NR³NR¹R² (wherein each of R¹, R², and R³ independently represents a hydrogen atom or a lower alkyl group), —NCO, —NCS, an epoxy group, and a vinyl group. The number of carbon atoms contained in a lower alkyl group is not particularly limited herein. It is generally approximately C1-C10, and preferably C1-C6.

Specific examples of a monomer compound having a functional group capable of immobilizing a physiologically active substance usable in the present invention may include acrylic acid, methacrylic acid, polyethylene glycol, polyethylene glycol acrylic acid monoester, and polyethylene glycol methacrylic acid monoester, but examples are not limited thereto.

A physiologically active substance is allowed to covalently bind to the biosensor surface obtained as described above via the aforementioned functional group, so as to immobilize the physiologically active substance on a metal surface or a metal film.

(4) Biosensor

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

Preferably, the substrate which constitutes the biosensor of the present invention is metal surface or metal film. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, more preferably between 0.5 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase; catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme or nucleic acid, an amino group, thiol group or the like of the physiologically active substance is covalently bound to a functional group located on a metal surface, so that the physiologically active substance can be immobilized on the metal surface.

A biosensor to which a physiologically active substance is immobilized as described above can be used to detect and/or measure a substance which interacts with the physiologically active substance.

Thus, the present invention provides a method of detecting and/or measuring a substance interacting with the physiologically active substance immobilized to the biosensor of the present invention, to which a physiologically active substance is immobilized, wherein the biosensor is contacted with a test substance.

As such a test substance, for example, a sample containing the above substance interacting with the physiologically active substance can be used.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

The biosensor of the present invention is preferably formed in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface,

wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

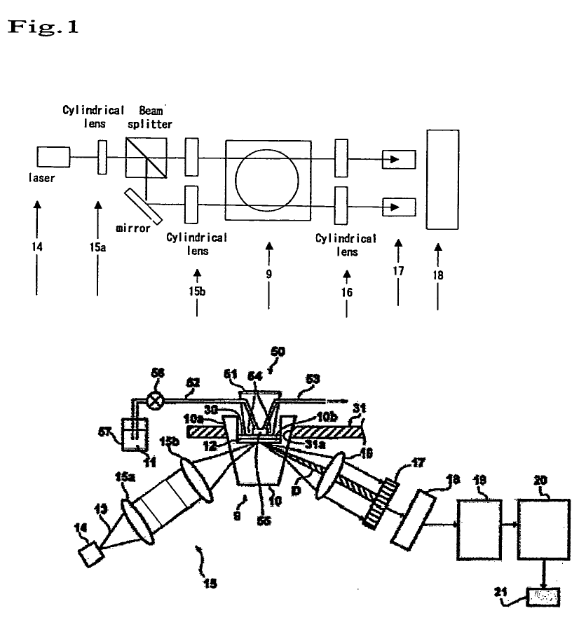

In the present invention, more specifically, a surface plasmon resonance measurement device shown in FIGS. 1 to 32 of Japanese Patent Laid-Open No. 2001-330560, and a surface plasmon resonance device shown in FIGS. 1 to 15 of Japanese Patent Laid-Open No. 2002-296177, can be preferably used. All of the contents as disclosed in Japanese Patent Laid-Open Nos. 2001-330560 and 2002-296177 cited in the present specification are incorporated herein by reference as a part of the disclosure of this specification.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

The device shown in FIG. 1 (the surface plasmon resonance measurement device of the present invention) was used in the following experiments. The details of the device shown in FIG. 1 are described in Japanese Patent Application Laid-Open (Kokai) No. 2003-254906.

### Example 1

### Production of Measurement Chip (The Present Invention)

(1) Production of Hydrophobic Film

The dielectric block of the present invention, which had been coated with gold via evaporation resulting in a metal film with a thickness of 50 nm, was treated with a Model-208 UV-ozone cleaning system (TECHNOVISION INC.) for 30 minutes. Thereafter, 5 μl of a methyl ethyl ketone solution containing 1 mg/ml the compound represented by P-1 or P-2 as shown below was added thereto, such that it was allowed to come into contact with the metal film. The resultant was then left at rest at 25° C. for 15 minutes. Thereafter, it was dried under reduced pressure at 40° C. for 2 hours. 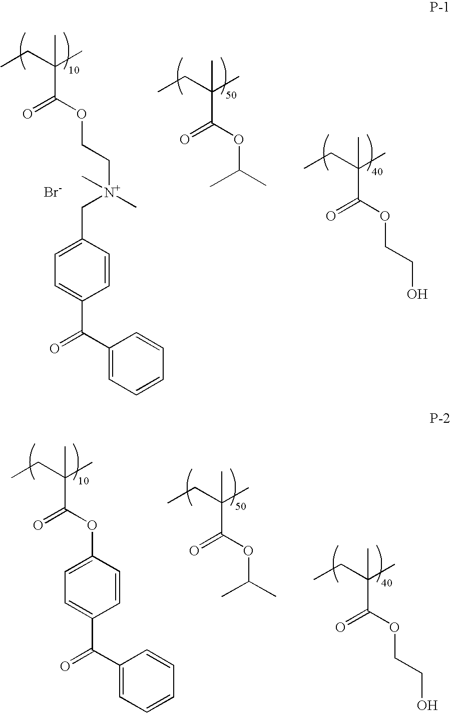
(2) Production of Two-Split Surface

10 μl of an aqueous solution containing 10% by mass of acrylic acid was added to each of the samples obtained in (1) above (namely, a sample obtained using compound P-1 and a sample obtained using compound P-2). Thereafter, a half of a measurement region was shaded with a metal mask, and it was then treated with a Model-208 UV-ozone cleaning system (TECHNOVISION INC.) for 30 minutes. The resultant was then washed with water. Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the reaction product, and the mixture was then left at rest at 25° C. for 30 minutes. The resultant product was washed with ethanol 5 times, and 20 μl of an ethanol solution containing 10 mM biotin-LC-amine (manufactured by PIERCE) was added thereto. The mixture was then left at 25° C. for 20 minutes. Thereafter, the resultant product was washed with ethanol 5 times, with a mixed solvent consisting of ethanol and water once, and then with water 5 times. At the same time, a portion exposed to light was shaded with a metal mask having a pattern opposite to that of the aforementioned metal mask, and the same light exposure as stated above was carried out. Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the reaction product, and the mixture was then left at rest at 25° C. for 30 minutes. Thereafter, the resultant product was washed with ethanol 5 times. Thereafter, 20 μl of an ethanol solution containing 1 M ethanolamine was added thereto, and the mixture was then left at rest at 25° C. for 20 minutes. Thereafter, the resultant product was washed with ethanol 5 times, with a mixed solvent consisting of ethanol and water once, and then with water 5 times. The obtained chip is called a light two-split surface chip.

### Comparative Example 1

### Production of Two-Split Surface by Contact

A stamp that was to be allowed to come into contact with a half of the measurement unit of a dielectric block used in measurement was produced by PDMS. The surface thereof was treated with plasma ozone, so as to keep solution wettability.

The dielectric block of the present invention, which had been coated with gold via evaporation resulting in a gold film with a thickness of 50 nm, was treated with a Model-208 UV-ozone cleaning system (TECHNOVISION INC.) for 30 minutes for cleaning. Thereafter, 5 μl of a methyl ethyl ketone solution containing 1 mg/ml PMMA was added thereto, such that it was allowed to come into contact with the metal film. The mixture was then left at rest at 25° C. for 15 minutes. The thickness of the obtained PMMA film was 20 nm.

1 N NaOH aqueous solution was added to the reaction product, such that it was allowed to come into contact with the above-described PMMA film. The resultant was then left at rest at 60° C. for 5 hours. Thereafter, the resultant product was washed with water 3 times. By this treatment, a carboxyl group was introduced into the surface of the PMMA film.

An ethanol solution containing 400 mM 1-ethyl-2,3-dimethylaminopropylcarbodiimide was mixed with an ethanol solution containing 100 mM pentafluorophenol at a ratio of 1:1, and 100 μl of the mixed solution was then allowed to contact with the aforementioned surface of the PMMA film into which a carboxyl group had been introduced. The resultant was left at rest at 25° C. for 30 minutes. Thereafter, the reaction product was washed with ethanol 5 times.

Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the thus obtained sample, and the mixture was then left at rest at 25° C. for 30 minutes. The resultant product was washed with ethanol 5 times. Thereafter, a stamp that had been immersed in an ethanol solution containing 10 mM biotin-LC-amine (manufactured by PIERCE) was allowed to come into contact with the resultant product for 20 minutes. Thereafter, the stamp was removed, and 40 μl of an ethanol solution containing 1 M ethanolamine was added thereto, followed by leaving at 25° C. for 20 minutes. Thereafter, the resultant product was washed with ethanol once, and a division wall was removed. The product was then washed with ethanol 5 times, with a mixed solvent consisting of ethanol and water once, and then with water 5 times. This sample is called a PMMA contact treatment chip.

### Test Example 1

(1) Evaluation of Two-Split Surface (Measurement of Non-Specific Adsorption)

100 μl of an HBS-EP solution containing 1% by weight of DMSO (dimethyl sulfoxide) was added to each of the light two-split surface chip produced in Example 1 and the PMMA contact treatment chip produced in Comparative example 1. A baseline was measured for 1 minute, and the obtained point was defined as a start point. Thereafter, the solution was exchanged with HBS-EP solution containing 100 μg/ml bovine serum albumin (HBS-EP solution) and 1% by weight of DMSO (dimethyl sulfoxide), and measurement was carried out. The HBS-EP solution consisted of 0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20. The point obtained after leaving for 15 minutes was defined as an end point, and the measurement was terminated. The value of a measurement plane and that of a reference plane were subtracted from the value, and the value of the start point was then subtracted from that of the end point. The obtained value was defined as NSB. Since the measurement plane having a surface modified with a biotin derivative did not interact with bovine serum albumin, NSB is preferably close to 0 (zero).

(2) Evaluation of Two-Split Surface (Measurement of Binding)

100 μl of an HBS-EP solution containing 1% by weight of DMSO (dimethyl sulfoxide) was added to each of the light two-split surface chip produced in Example 1 and the PMMA contact treatment chip produced in Comparative example 1. A baseline was measured for 1 minute, and the obtained point was defined as a start point. Thereafter, the solution was exchanged with an HBS-EP solution containing 0.1 μg/ml avidin, 100 μg/ml bovine serum albumin, and 1% by weight of DMSO (dimethyl sulfoxide), and measurement was carried out. The point obtained after leaving for 15 minutes was defined as an end point, and the measurement was terminated. The value of a measurement plane and that of a reference plane were subtracted from the value, and the value of the start point was then subtracted from that of the end point. The obtained value was defined as BIND.

(3) Evaluation Results

The results of the above-described measurements are shown in Table 1.

TABLE 1

| | NSB (RU) | BIND (RU) | Remarks |
|---|---|---|---|
| P-1 two-split surface | 6 RU | 946 RU | The present invention |
| P-2 two-split surface | 4 RU | 945 RU | The present invention |
| PMMA contact | 254 RU | 1,380 RU | Comparative example |
| treatment chip | | | |

When a two-split surface was produced by contact, a thin film existing on gold was destroyed and the gold surface appeared. As a result, non-specific adsorption was deteriorated. This means that the value caused by such non-specific adsorption is loaded on the actual binding value. Thus, this results in deterioration in measurement accuracy. Accordingly, it is found that the purpose can be achieved by the configuration of the present invention.

### Example 2

### Production of Measurement Chip (The Present Invention)

(1) Production of Hydrophobic Film

The dielectric block of the present invention, which had been coated with gold via evaporation resulting in a metal film with a thickness of 50 nm, was treated with a Model-208 UV-ozone cleaning system (TECHNOVISION INC.) for 30 minutes. Thereafter, 5 μl of a methyl ethyl ketone solution containing 1 mg/ml polystyrene was added thereto, such that it was allowed to come into contact with the metal film. The resultant was then left at rest at 25° C. for 15 minutes. Thereafter, the reaction product was dried under reduced pressure at 40° C. for 2 hours.

(2) Production of Two-Split Surface

10 μl of an aqueous solution containing 10% by mass of 0-(2-azidoethyl)-0-[2-(diglycosyl-amino)ethyl]heptaethylene glycol (Azi-PEG-acid (n=8)) (manufactured by Fluka) was added to the sample obtained in (1) above. Thereafter, a half of a measurement region was shaded with a metal mask, and it was then treated with a Model-208 UV-ozone cleaning system (TECHNOVISION INC.) for 30 minutes. The resultant was then washed with water. Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the reaction product, and the mixture was then left at rest at 25° C. for 30 minutes. The resultant product was washed with ethanol 5 times, and 20 μl of an ethanol solution containing 10 mM biotin-LC-amine (manufactured by PIERCE) was added thereto. The resultant was then left at 25° C. for 20 minutes. Thereafter, the resultant product was washed with ethanol 5 times, with a mixed solvent consisting of ethanol and water once, and then with water 5 times. At the same time, a portion exposed to light was shaded with a metal mask having a pattern opposite to that of the aforementioned metal mask, and the same light exposure as stated above was carried out. Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the reaction product, and the mixture was then left at rest at 25° C. for 30 minutes. Thereafter, the resultant product was washed with ethanol 5 times. Thereafter, 20 μl of an ethanol solution containing 1 M ethanolamine was added thereto, and the mixture was then left at rest at 25° C. for 20 minutes. Thereafter, the resultant product was washed with ethanol 5 times, with a mixed solvent consisting of ethanol and water once, and then with water 5 times. The obtained chip is called a light two-split surface chip.

### Comparative Example 2

### Production of Two-Split Surface by Contact

A stamp that was to be allowed to come into contact with a half of the measurement unit of a dielectric block used in measurement was produced by PDMS. The surface thereof was treated with plasma ozone, so as to keep solution wettability.

The dielectric block of the present invention, which had been coated with gold via evaporation resulting in a gold film with a thickness of 50 nm, was treated with a Model-208 UV-ozone cleaning system (TECHNOVISION INC.) for 30 minutes for cleaning. Thereafter, 5 μl of a methyl ethyl ketone solution containing 1 mg/ml PMMA was added thereto, such that it was allowed to come into contact with the metal film. The mixture was then left at rest at 25° C. for 15 minutes. The thickness of the obtained PMMA film was 20 nm.

1 N NaOH aqueous solution was added to the reaction product, such that it was allowed to come into contact with the above-described PMMA film. The mixture was then left at rest at 60° C. for 5 hours. Thereafter, the resultant product was washed with water 3 times. By this treatment, a carboxyl group was introduced into the surface of the PMMA film.

An ethanol solution containing 400 mM 1-ethyl-2,3-dimethylaminopropylcarbodiimide was mixed with an ethanol solution containing 100 mM pentafluorophenol at a ratio of 1:1, and 100 μl of the mixed solution was then allowed to contact with the aforementioned surface of the PMMA film into which a carboxyl group had been introduced. The mixture was left at rest at 25° C. for 30 minutes. Thereafter, the reaction product was washed with ethanol 5 times.

Subsequently, 100 μl of a mixed solution prepared by mixing an ethanol solution containing 400 mM 1-ethyl-2,3-dimethylaminopropylcarbodiimide with an ethanol solution containing 100 mM pentafluorophenol at a ratio of 1:1 was added to the thus obtained sample, and the resultant was then left at rest at 25° C. for 30 minutes. The resultant product was washed with ethanol 5 times. Thereafter, a stamp that had been immersed in an ethanol solution containing 10 mM biotin-LC-amine (manufactured by PIERCE) was allowed to come into contact with the resultant product for 20 minutes. Thereafter, the stamp was removed, and 40 μl of an ethanol solution containing 1 M ethanolamine was added thereto, followed by leaving at 25° C. for 20 minutes. Thereafter, the resultant product was washed with ethanol once, and a division wall was removed. The product was then washed with ethanol 5 times, with a mixed solvent consisting of ethanol and water once, and then with water 5 times. This sample is called a PMMA contact treatment chip.

### Test Example 2

(1) Evaluation of Two-Split Surface (Measurement of Non-Specific Adsorption)

100 μl of an HBS-EP solution containing 1% by weight of DMSO (dimethyl sulfoxide) was added to each of the light two-split surface chip produced in Example 2 and the PMMA contact treatment chip produced in Comparative example 2. A baseline was measured for 1 minute, and the obtained point was defined as a start point. Thereafter, the solution was exchanged with an HBS-EP solution containing 100 μg/ml bovine serum albumin (HBS-EP solution) and 1% by weight of DMSO (dimethyl sulfoxide), and measurement was carried out. The HBS-EP solution consisted of 0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20. The point obtained after leaving for 15 minutes was defined as an end point, and the measurement was terminated. The value of a measurement plane and that of a reference plane were subtracted from the value, and the value of the start point was then subtracted from that of the end point. The obtained value was defined as NSB. Since the measurement plane having a surface modified with a biotin derivative did not interact with bovine serum albumin, NSB is preferably close to 0 (zero).

(2) Evaluation of Two-Split Surface (Measurement of Binding)

100 μl of an HBS-EP solution containing 1% by weight of DMSO (dimethyl sulfoxide) was added to each of the light two-split surface chip produced in Example 2 and the PMMA contact treatment chip produced in Comparative example 2. A baseline was measured for 1 minute, and the obtained point was defined as a start point. Thereafter, the solution was exchanged with an HBS-EP solution containing 0.1 μg/ml avidin, 100 μg/ml bovine serum albumin, and 1% by weight of DMSO (dimethyl sulfoxide), and measurement was carried out. The point obtained after leaving for 15 minutes was defined as an end point, and the measurement was terminated. The value of a measurement plane and that of a reference plane were subtracted from the value, and the value of the start point was then subtracted from that of the end point. The obtained value was defined as BIND.

(3) Evaluation Results

The results of the above-described measurements are shown in Table 2.

TABLE 2

| | NSB (RU) | BIND (RU) | Remarks |
|---|---|---|---|
| Azi-PEG-acid (n = 8) | 4 RU | 935 RU | The present invention |
| two-split surface | | | |
| PMMA contact | 259 RU | 1,360 RU | Comparative example |
| treatment chip | | | |

When a two-split surface was produced by contact, a thin film existing on gold was destroyed and the gold surface appeared. As a result, non-specific adsorption was deteriorated. This means that the value caused by such non-specific adsorption is loaded on the actual binding value. Thus, this results in deterioration in measurement accuracy. Accordingly, it is found that the purpose can be achieved by the configuration of the present invention.

### EFFECTS OF THE INVENTION

According to the present invention, it becomes possible to provide a biosensor, wherein at least two types of surfaces are patterned without impairing the sensor surface and non-specific adsorption is thereby suppressed.

1. A biosensor comprising a substrate coated with a hydrophobic compound having a photoactive group, or a substrate which is coated with a hydrophobic polymer and is further modified with a compound having a photoactive group.

2. The biosensor of claim 1, wherein at least two types of surfaces are patterned by light irradiation on a substrate.

3. The biosensor of claim 1, wherein the substrate is a metal surface or metal film.

4. The biosensor of claim 1, wherein the coating thickness of the hydrophobic compound having a photoactive group or the hydrophobic polymer is between 0.1 nm and 500 nm.

5. The biosensor of claim 1, which has a functional group capable of immobilizing a physiologically active substance on the outermost surface of the substrate.

6. The biosensor of claim 1, wherein the compound having a photoactive group has a functional group capable of immobilizing a physiologically active substance.

7. The biosensor of claim 5, wherein the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R²and R³ independently represents a hydrogen atom or lower alkyl group), —NCO, —NCS, an epoxy group, or a vinyl group.

8. The biosensor of claim 5, which has a functional group capable of immobilizing a physiologically active substance in a certain region on the outermost surface of the substrate, which has been patterned by light irradiation.

9. The biosensor of claim 1, which is used in surface plasmon resonance analysis.

10. The biosensor of claim 1, which is formed in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface,
wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

11. A method for producing the biosensor of claim 1, which comprises a step of coating a substrate with a hydrophobic compound having a photoactive group and a step of applying light to the substrate.

12. A method for producing the biosensor of claim 1, which comprises a step of coating a substrate with a hydrophobic compound having a photoactive group, a step of allowing a monomer compound having a functional group capable of immobilizing a physiologically active substance to come into contact with the substrate, and a step of applying light to only certain region of the substrate with making patterning so as to bind said monomer compound.

13. A method for producing the biosensor of claim 1, which comprises a step of allowing a substrate coated with a hydrophobic polymer to come into contact with a compound having a photoactive group.

14. A method for producing the biosensor of claim 1, which comprises a step of allowing a compound having a photoactive group to come into contact with a substrate coated with a hydrophobic polymer, and a step of applying light to only certain region of the substrate with making patterning.

15. The biosensor of claim 1, wherein a physiologically active substance is bound to the surface by covalent bonding.

16. The biosensor of claim 1, wherein a physiologically active substance is bound by using a compound having a photoactive group.

17. The biosensor of claim 15, wherein at least one measurement unit to which a physiologically active substance or a substance interacting therewith binds, and one reference unit that does not have a physiologically active substance or a substance interacting therewith, exist on a single plane.

18. A method for immobilizing a physiologically active substance on a biosensor, which comprises a step of allowing a physiologically active substance to come into contact with the biosensor of claim 1, so as to allow said physiologically active substance to bind to the surface of said biosensor via a covalent bond.

19. A method for detecting or measuring a substance interacting with a physiologically active substance, which comprises a step of allowing a test substance to come into contact with the biosensor of claim 1 to the surface of which the physiologically active substance binds via a covalent bond.

20. The method of claim 19, wherein the substance interacting with the physiologically active substance is detected or measured by surface plasmon resonance analysis.
